(19) United States
(12) Plant Patent Application Publication
(10) Pub. No.: US 2011/0067152 P1
(43) Pub. Date: Mar. 17, 2011

(54) Apple tree named "Delicia'

(50) ;

(76) Inventors: Roduit Aebischer C., Manosque (FR); Roduit Aebischer S., Manosque (FR)

(21) Appl. No.: 12/807,704

(22) Filed: Sep. 9, 2010

Related U.S. Application Data
(60) Provisional application No. 61/276,680, filed on Sep. 14, 2009.

(51) Int. Cl. A01H 5/00

(52) U.S. Cl. PLT/162

(57) ABSTRACT

‘Delicia’ is a new and distinct variety of apple tree (Malus domestica Borkh). A mutation of ‘Gala’ (not patented), ‘Delicia’ is characterized by its sparse light orange overcolor.

### CROSS REFERENCE TO RELATED APPLICATIONS

This application claims priority under 35 U.S.C. 119 of copending provisional application Ser. No. 61/276,680 filed Sep. 13, 2010.

### LATIN NAME

Malus domestica Borkh

### COMMON NAME

Apple

### VARIETY DENOMINATION ‘Delicia’

### BACKGROUND OF THE VARIETY

‘Delicia’ is a new and distinct variety of apple tree Malus domestica Borkh. This new variety is a mutation of ‘Gala’ (not patented), and is characterized by its sparse light orange overcolor. ‘Delicia’ was first observed at Manosque, France in 2001, and was asexually reproduced by grafting at the same location in 2002. The variety has been observed to remain true to type over successive asexually propagated generations.

‘Delicia’ is the subject of European Community Plant Variety Rights Grant No. 20400 (Application No. 2002/1818).

### BRIEF DESCRIPTION OF THE VARIETY

‘Delicia’ was first selected for its uniquely colored fruit. It is distinguished from other known Gala-type varieties by its sparse, light orange overcolor. The distinctive characteristics are shown in Table 1 below.

| | | |
|---|---|---|
| Fruit Characteristic | ‘Gala’ | ‘Delicia’ |
| Amount of over colour | Medium | Absent or very little |
| Pattern of over colour of skin | Solid flush with stripes | Washed out |
| Over colour | Red | Orange |
| Intensity of over colour | Medium | Light |

### BRIEF DESCRIPTION OF THE PHOTOGRAPHS

FIG. 1 shows a tree of ‘Delicia’;

FIG. 2 shows the upper surface of leaves of ‘Delicia’;

FIG. 3 shows the lower surface of leaves of ‘Delicia’; and

FIG. 4 shows whole and sectioned fruit of ‘Delicia’.

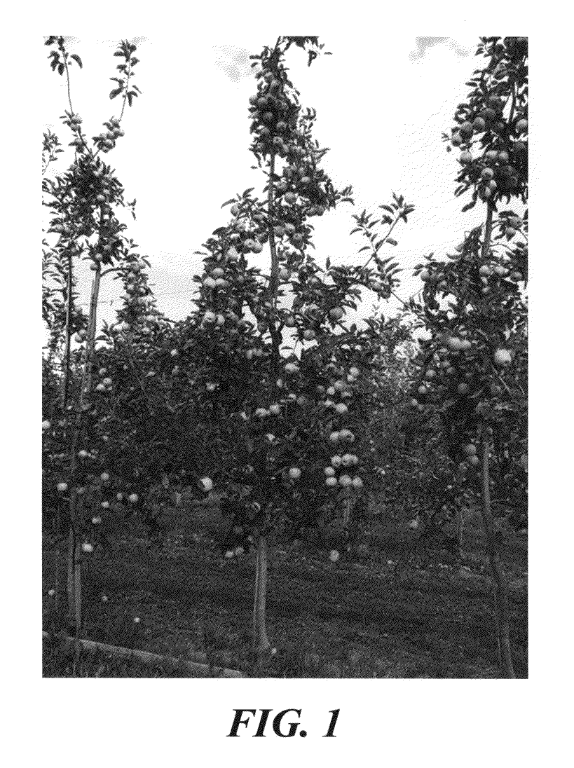

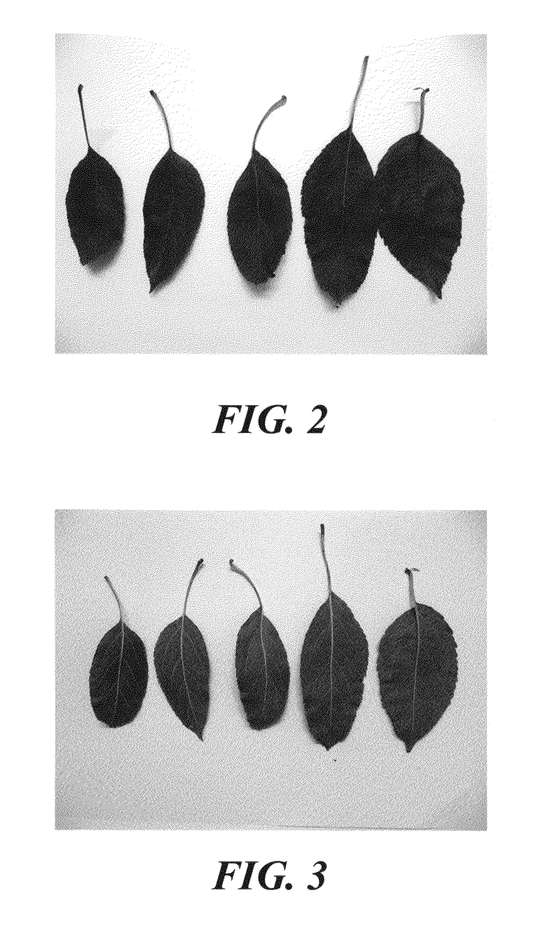

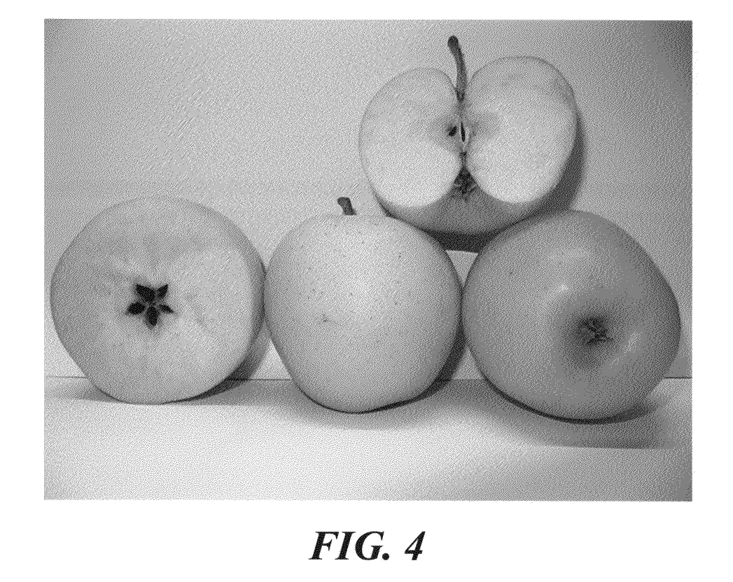

### DETAILED BOTANICAL DESCRIPTION OF THE VARIETY

The following-detailed botanical description is based on observations made during the 2006 and 2010 growing seasons at La Pugère experiment station (France) of trees planted in 2005 and grown on ‘9EMLA’ rootstock (not patented). It should be understood that the characteristics described will vary somewhat depending upon cultural practices and climatic conditions, and will vary with location and season. Quantified measurements are expressed as an average of measurements taken from a number of individual plants of the new variety. The measurements of any individual plant or any group of plants of the new variety may vary from the stated average.
Tree:
   Vigour.—medium (similar to ‘Golden Delicious’, not patented).
   Type.—ramified.
   Habit.—spreading.
   Height.—medium-small.
   Trunk diameter.—medium (16 to 20 cm).
   Bark color.—Light grey.
   Bark texture.—Smooth.
Dormant one-year-old shoot:
   Pubescence (on upper half of shoot).—weak.
   Length.—30 to 40 cm.
   Thickness.—medium (similar to ‘Jonagold’, not patented).
   Length of internode.—medium.
   Number of lenticels.—many (similar to ‘Mutsu’, not patented).
Branch:
   Length.—medium.
   Diameter.—medium.
   Crotch angle.—wide.
   Bark color.—grey.
   Lenticel length.—medium long.
   Lenticel color.—black.
   Number of lenticels per cm².—numerous.
Unopened flower:
   Colour.—light pink.
Flower:
   Diameter of flower with petals pressed into horizontal position.—medium-small (3.1 cm).
Petals:
   Relative position of margins.—free.
   Margin shape.—smooth.
Leaf:
   Attitude in relation to shoot.—upwards.
Leaf blade:
   Length.—long.
   Width.—medium.
   Ratio length/width.—large.
   Shape of incisions of margin.—crenate.
   Color-upper surface.—olive green.
   Color-lower surface.—green.
Petiole:
   Length.—medium.
Fruit:
   Size.—medium (similar to ‘Cox's Orange Pippin’, not patented).
   Diameter.—about 6.5 to 7.5 cm.
   Ratio height to width.—medium, about 1:1.
   Position of maximum width.—towards stalk.
   Weight.—about 167 to 210 g.
   Shape.—truncate conical.
   Ribbing.—medium.
   Crowning at calyx end.—medium.
   Aperture of eye.—closed.
   Size of eye.—medium.
   Length of sepal.—long.
   Depth of eye basin.—deep.
   Width of eye basin.—medium.
   Thickness of stalk.—medium.
   Length of stalk.—long.
   Color of stalk.—yellow brown.
   Depth of stalk.—cavity deep.
   Width of stalk.—cavity medium.
   Bloom of skin.—absent or very weak.
   Greasiness of skin.—absent or very weak.
   Ground colour (if visible).—yellow.
   Amount of over colour.—absent or very low.
   Over colour.—orange.
   Intensity of over colour.—light.
   Pattern of over colour of skin.—washed out.
   Amount of russet around eye basin.—absent or very low.
   Amount of russet on cheeks.—absent or very low.
   Amount of russet around stalk cavity.—low.
   Size of lenticels.—small.
   Firmness of the flesh (measurement with penetrometer).—medium.
   Colour of the flesh.—yellowish, satin white.
   Flesh texture.—juicy, fine, crisp.
   Aroma.—aromatic, similar to ‘Gala’.
Fruit in cross-section:
   Aperture of locules (median through locules).—partly open.
Seeds:
   Quantity per fruit.—7 to 9.
   Color.—dark brown.
   Time of beginning of flowering (10% open flowers).—medium (similar to ‘Jonagold’).
   Time of maturity for consumption.—medium (similar to ‘Gala’); August 18 in La Pugère, France (2010 growing season).
   Number of picks.—3 .
   Yield.—68 to 118 fruits per tree.
Disease/pest resistance or susceptibility: none noted (similar to ‘Gala’).
   Market.—fresh.

1. We claim a new and distinct apple tree named ‘Delicia’ substantially as illustrated and described herein.
